(19) United States
(12) Patent Application Publication
(10) Pub. No.: US 2025/0207164 A1
(43) Pub. Date: Jun. 26, 2025

(54) METHOD AND KIT FOR 3'-END MODIFICATION OF NUCLEIC ACIDS

(71) Applicant: Cheng-Yao CHEN, Hsinchu (TW)

(72) Inventor: Cheng-Yao CHEN, Hsinchu (TW)

(21) Appl. No.: 18/852,496

(22) PCT Filed: May 26, 2023
(86) PCT No.: PCT/US2023/023708

§ 371 Date: Sep. 29, 2024

Related U.S. Application Data
(60) Provisional application No. 63/346,416, filed on May 27, 2022.

(51) Int. Cl. C12P 19/34; C12N 9/12

(52) U.S. Cl. CPC C12P 19/34; C12N 9/1252; C12Y 207/07007

(57) ABSTRACT

Provided is a method for introducing a modification to a 3′-end of a polynucleotide, including coupling a detectable label or a tag to the 3-end of the polynucleotide. Also provided is a kit for modifying a polynucleotide at the 3-end of the polynucleotide. The kit includes a polymerase, a nucleotide with a reactive moiety, and a desired molecule to be coupled to the nucleotide.

### BACKGROUND

### Technical Field

The present disclosure relates to modifications of nucleic acids, more particularly to modifications at the 3′-end of nucleic acids.

### Description of Related Art

Nucleic acid modifications, e.g., oligonucleotides labeled with unique chemical tags at the 5′-end or the 3′-end, have been widely used in molecular biology in applications such as DNA microarray, next-generation sequencing, and molecular diagnostics. These labeled nucleic acids are essential tools for direct sequence-specific detection or target sequence enrichment of DNA or RNA molecules. For instance, nucleic acid probes (NAPs) generated through covalent conjugation of fluorophores and quenchers have become indispensable for sequence-specific detection and quantitation of nucleic acid molecules and amplicons of clinical DNA/RNA samples. These NAPs can help in the identification of clinical microorganisms and genetic diseases. Also, gene probes are commonly used in various nucleic acid blotting and in situ hybridization (ISH) techniques for the detection of nucleic acid sequences in food industry and in environmental, biomedical, and veterinary diagnostic applications. In addition to the clinical utilities, the labeled tags of nucleic acids can also be used as an anchor for attaching nucleic acids to solid supports or molecules to facilitate immobilization or target delivery.

Presently, a plethora of chemical or enzymatic methods are available to generate nucleic acid probes such as those labeled with fluorophores, enzymes and radioactive phosphates, or nucleotides modified with digoxigenin or biotin. However, conventional chemical methods for preparing labeled nucleic acids require complicated chemical modifications at the 5′-end or 3′-end to produce nucleic acids with a regiospecific functional group for subsequent labeling or conjugation reaction. These methods are generally tedious and inefficient.

Alternatively, enzymatic methods for nucleic acid labeling are simple and straightforward. For example, DNA nick translation using DNase I and DNA polymerase I is widely adapted to label DNA for use as a hybridization probe. In this method, DNase I needs to first cut one strand of DNAs and expose 5′-phosphoryl and 3′-hydroxyl (3′-OH) ends of DNAs. The labeled nucleotides (radioactive or fluorescent) can then be incorporated into the nicked DNA strand by DNA polymerase I (Pol I). At the end of DNA synthesis, the newly synthesized DNA strands containing labeled nucleotides are obtained. Likewise, terminal deoxynucleotidyl transferase (TdT) is another DNA polymerase often used to label DNA probes for RACE (rapid amplification of cDNA ends) and TUNEL (terminal deoxynucleotidyl transferase dUTP nick-end labeling) assays, and also serves as a method for adding 3′ overhangs to DNA fragments to facilitate DNA cloning.

However, both Pol I- and TdT-based enzymatic methods for labeling nucleic acids have inherent limitations. For example, DNA nick translation relies on Pol I to incorporate base-labeled nucleotides that requires a complementary DNA template for DNA replication. As a result, this method can only be used for labeling double-stranded (ds) DNAs and therefore is unsuitable for labeling at the 5′-end or 3′-end of DNAs.

Unlike Pol I, the TdT enzyme can add a base-labeled nucleotide to the 3′-end of single-stranded (ss) DNA or blunt-end, double-stranded DNA. However, the TdT enzyme cannot efficiently utilize the 3′-modified nucleotide for DNA elongation reaction.

Furthermore, both Pol I and TdT enzymes used for nucleic acid-labeling methods exhibit a strong preference for certain types of modified nucleotides with a distinct labeled molecule. The bias of nucleotide utilization greatly restrains both the Pol I- and TdT-based nucleic acid labeling methods for broader applications of nucleic acid probes (NAPs).

Therefore, there remains an unmet need to label nucleic acids at the 3′-end, e.g., a nucleic acid labeling method for efficiently introducing nucleotides with modifications or labeling molecules at the 3′-end of nucleic acids.

### SUMMARY

The present disclosure provides a method for efficiently modifying or labeling the 3′-end of nucleic acids or polynucleotides. The method of the present disclosure deploys a pair of functional moieties or chemical molecules that can stably or covalently couple with each other, thereby introducing a specific modification or label to the 3′-end of nucleic acids or polynucleotides. For example, one component of the paired functional moieties or chemical molecules is labeled on a nucleobase or the 3′-hyodroxyl (3′-OH) group of a nucleotide, which is incorporated by a polymerase to the 3′-end of the target nucleic acid or polynucleotide. The resulting target nucleic acid or polynucleotide contains the first functional moiety or chemical molecule at the 3′-end, which can readily react with the other component of the paired moieties or molecules carrying a desired modification or label. Consequently, the reaction between the paired functional moieties forms a stable or covalent linkage, such that the target nucleic acid or polynucleotide can be modified or labeled with the desired molecule at the 3′-end of the target nucleic acid or polynucleotide.

In at least one embodiment, the method provided by the present disclosure comprises modifying a natural or synthetic deoxyribonucleic acid (DNA) at the 3′-end. FIG. 1 shows a schematic diagram depicting an exemplary method of introducing a 3′-modification to a target nucleic acid. In some embodiments, the method provided by the present disclosure comprises providing a polynucleotide including a 3′-end nucleotide (e.g., “Nucleotide” as illustrated in FIG. 1) having a reactive moiety (e.g., “M1” as illustrated in FIG. 1); and exposing the polynucleotide to a desired molecule (e.g., “Label” as illustrated in FIG. 1) having a corresponding functional moiety (e.g., “M2” as illustrated in FIG. 1) capable of reacting with the reactive moiety to form a linkage, thereby coupling the desired molecule to the 3′-end of the polynucleotide. In some embodiments, the desired molecule has a label moiety (e.g., “Label” as illustrated in FIG. 1) being introduced into the polynucleotide to form a labeled polynucleotide.

In at least one embodiment, the method provided by the present disclosure further comprises preparing the polynucleotide by a template-independent enzymatic nucleic acid synthesis. In some embodiments, the template-independent enzymatic nucleic acid synthesis comprises employing a DNA polymerase, an RNA polymerase, or a functionally equivalent enzyme thereof. In some embodiments, the DNA polymerase of the template-independent enzymatic nucleic acid synthesis is an A family DNA polymerase, a B family DNA polymerase, or an X family DNA polymerase. In at least one embodiment, the B family DNA polymerase is a Thermococcaceae DNA polymerase. In at least one embodiment, the B family DNA polymerase is a Thermococcus DNA polymerase or a Prococcus DNA polymerase. In at least an embodiment, the B family DNA polymerase is selected from the group consisting of a B family DNA polymerase of Thermococcus kodakarensis (Kodl), a B family DNA polymerase of Pyrococcus furiosus (Pfu), a B family DNA polymerase of Thermococcus litoralis (Vent), a B family DNA polymerase of Thermococcus sp. 9° N (9° N), and a B family DNA polymerase of Thermococcus gorgonarius (Tgo). In some embodiments, the DNA polymerase of the template-independent enzymatic nucleic acid synthesis is a modified DNA polymerase.

In at least one embodiment of the present disclosure, the template independent enzymatic nucleic acid synthesis is performed at a reaction temperature of from 10° C. to 100° C., such as 10° C. to 90° C., 20° C. to 90° C., 30° C. to 90° C., 20° C. to 80° C., 30° C. to 80° C., 40° C. to 80° C., 30° C. to 70° C., 40° C. to 70° C., or 50° C. to 70° C.

In some embodiments, the method provided by the present disclosure further comprises preparing the polynucleotide in a solution phase. In other embodiments, the method provided by the present disclosure comprises preparing the polynucleotide in a solid phase, e.g., providing an initiator attached to a solid support. In some embodiments, the solid support is selected from the group consisting of a particle, a polymer, a bead, a resin, a slide, a chip, an array surface, a membrane, a flow cell, a well, a matrix, a chamber, a microfluidic chamber, a channel, a microfluidic channel, and a gel.

In at least one embodiment, the method provided by the present disclosure further comprises providing an endonuclease to enzymatically release the polynucleotide from the initiator. In some embodiments, the endonuclease recognizes the 3′-penultimate nucleotide of the initiator and cleaves a linkage bond between the 3′-end nucleotide of the initiator and the polynucleotide, between the 3′-penultimate nucleotide and a 3′-antepenultimate nucleotide of the initiator, between a 3′-antepenultimate nucleotide and a 3′-preantepenultimate nucleotide of the initiator, or between a 3′-preantepenultimate nucleotide and a 3′-propreantepenultimate nucleotide of the initiator.

In at least one embodiment of the present disclosure, the endonuclease is derived from Thermococcus barophilus (Tba), Pyrococcus furiosus (Pfu), Methanosarcina acetivorans (Mac), Pyrococcus abyssi (Pab), Thermococcus kodakarensis (Tko), Thermococcus gammatolerans (Tga), or Bacillus subtilis (Bsu).

In at least one embodiment of the present disclosure, the 3′-end nucleotide is a natural nucleotide, a nucleotide analogue, or an abasic (apurinic/apyrimidinic) nucleotide. In some embodiments, the 3′-end nucleotide is a ribonucleotide, a deoxyribonucleotide, or a xeno-nucleotide. In some embodiments, the reactive moiety is linked to the 2′-carbon or 3′-carbon of the nucleosugar, or the nucleobase of the 3′-end nucleotide.

In at least one embodiment of the present disclosure, the corresponding functional moiety reacts with the reactive moiety via a bioorthogonal reaction. In some embodiments, the bioorthogonal reaction is click conjugation, oxime/hydrazine formation, Staudinger ligation, tetrazine ligation, or quadricyclane ligation. In some embodiments, the click conjugation is selected from the group consisting of copper-catalyzed azide-alkyne cycloaddition (CuAAC), strain-promoted azide-alkyne cycloaddition (SPAAC), isocyanide-based click reaction, and inverse electron demand Diels-Alder reaction (IEDDA).

In at least one embodiment of the present disclosure, the reactive moiety is selected from the group consisting of an azido group, an alkynyl group, a triarylphosphinyl group, a cyclooctynyl group, a thiol group, an alkenyl group, a nitrone group, an aldehydyl group, a ketonyl group, a dienyl group, and a dienophilyl group.

In at least one embodiment of the present disclosure, the corresponding functional moiety is a functional group selected from the group consisting of an azido group, an alkynyl group, a triarylphosphinyl group, a cyclooctynyl group, a thiol group, an alkenyl group, a nitrone group, an aldehydyl group, a ketonyl group, a dienyl group, and a dienophilyl group.

In some embodiments, the bioorthogonal reaction is performed at a reaction temperature of from 10° C. to 100° C., such as 10° C. to 90° C., 10° C. to 80° C., 10° C. to 70° C., 10° C. to 60° C., 20° C. to 80° C., 20° C. to 70° C., 20° C. to 60° C., 20° C. to 50° C., 30° C. to 70° C., 30° C. to 60° C., 30° C. to 50° C., or 30° C. to 40° C. In some embodiments, the bioorthogonal reaction is performed for, e.g., 1 min, 5 min, 10 min, 20 min, 30 min, 40 min, 50 min, 1 hr, 2 hr, 5 hr, 10 hr, 12 hr, 16 hr, 24 hr, 36 hr, 48 hr or more.

In at least one embodiment, the desired molecule is molecularly recognizable through detection of visible light, fluorescence, photoluminescence, electrochemiluminescence, laser, irradiation, fluorescence resonance energy transfer, fluorogenic conformational change, or fluorescence quenching. In some embodiments, the desired molecule is a chemical compound, a fluorescent tag, a dye, a marker, a reporter, a quencher, an amine, an antigen, a ligand, a protein, an antibody, an antibody fragment, a peptide, a peptide analog, or a quantum dot.

In at least one embodiment, the method provided by the present disclosure further comprises a clean-up or enrichment step to remove unlabeled nucleic acids or polynucleotides, e.g., providing a protein possessing a 3′ to 5′ exonuclease activity to digest the nucleic acids or polynucleotides with an unsuccessful 3′-end nucleotide synthesis by a polymerase or an incomplete coupling reaction with a second reactive moiety.

In at least one embodiment, the present disclosure also provides a kit for modifying a polynucleotide at its 3′-end, which comprises a nucleotide having a reactive moiety, a polymerase, a desired labeling molecule, and a 3′ to 5′ exonuclease, wherein the polynucleotide is coupled with the desired labeling molecule at the 3′-end to form a labeled polynucleotide, which can be further enriched by the clean-up exonuclease described herein.

### BRIEF DESCRIPTION OF THE DRAWINGS

The present disclosure will become more readily appreciated and better understood by reference to the following descriptions, when taken in conjunction with the accompanying drawings.

FIG. 1 is a schematic diagram depicting an exemplary method of introducing a 3′-modification to a nucleic acid or polynucleotide and the enrichment of the labeled nucleic acid or polynucleotide.

FIGS. 2A and 2B show an example of labeling a Cy5-fluorescent dye/fluorophore to the 3′-end of a polynucleotide via the enzymatic synthesis of 3′-O-azidomethyl deoxynucleotide (3′-AZ-dNTP) to the 3′-end of the polynucleotide, followed by the azide-alkyne click conjugation reaction between the incorporated 3′-O-azidomethyl deoxynucleoside monophosphate (3′-AZ-dNMP) and the alkyne-modified Cy5-fluorescent dye moiety. FIGS. 2A and 2B are images from the same gel. While FIG. 2A depicts the gel electrophoresis result of the unlabeled and labeled polynucleotides visualized by staining with SYBR Gold dye, FIG. 2B illustrates the electrophoretic location of nucleic acids labeled with the Cy5-fluorescent dye after the enrichment step. Lane 1: the electrophoretic location of the target polynucleotide (45-mer single-stranded DNA); lane 2: the electrophoretic location of the target polynucleotide plus an incorporated 3′-AZ-dNMP at the 3′-end; and lane 3: the electrophoretic location of the polynucleotide plus an incorporated 3′AZ-dNMP coupled with a Cy5-fluorescent dye at the 3′-end after the enrichment step.

FIG. 3 shows an example of labeling a fluorescent quencher (BHQ1) to the 3′-end of a polynucleotide via the enzymatic synthesis of 3′-O-azidomethyl deoxynucleotide (3′-AZ-dNTP) to the 3′-end of the polynucleotide, followed by the azide-alkyne click conjugation reaction between the incorporated 3′-AZ-dNMP and the alkyne modified fluorescent quencher moiety. Lane 1: the electrophoretic location of the polynucleotide (45-mer single-stranded DNA); lane 2: the electrophoretic location of the polynucleotide plus an incorporated 3′-AZ-dNMP at the 3′-end; lane 3: the electrophoretic location of the polynucleotide plus an incorporated 3′-AZ-dNMP coupled with, or without, a fluorescent quencher at the 3′-end; and lane 4: the electrophoretic location of the polynucleotide plus an incorporated 3′-AZ-dNMP coupled with a fluorescent quencher at the 3′-end after the enrichment step.

FIGS. 4A and 4B show an example of labeling a Cy5-fluorescent dye/fluorophore to the 3′-end of a polynucleotide via the enzymatic synthesis of 3′-O-azidomethyl deoxynucleotide (3′-AZ-dNTP) to the 3′-end of the polynucleotide, followed by the azide-DBCO conjugation reaction between the incorporated 3′-AZ-dNMP and the DBCO-modified Cy5-fluorescent dye moiety. FIGS. 4A and 4B are images from the same gel. While FIG. 4A depicts the gel electrophoresis result of the unlabeled and labeled polynucleotides visualized by staining with the SYBR Gold dye, FIG. 4B illustrates the electrophoretic location of nucleic acids labeled with the Cy5-fluorescent dye at the 3′-end. Lane 1: the electrophoretic location of the target polynucleotide (45-mer single-stranded DNA); and lane 2: the electrophoretic location of the polynucleotide plus an incorporated 3′-AZ-dNMP conjugated with a Cy5-fluorescent dye at the 3′-end.

FIG. 5 shows an example of labeling a fluorescent quencher (BHQ1) to the 3′-end of a target polynucleotide via the enzymatic synthesis of 3′-O-azidomethyl deoxynucleotide (3′-AZ-dNTP) to the 3′-end of the polynucleotide, followed by the azide-DBCO conjugation reaction between the incorporated 3′-AZ-dNMP and the DBCO-modified fluorescent quencher moiety. In this figure, lane 1 shows the electrophoretic location of the polynucleotide (45-mer single-stranded DNA); lane 2 shows the electrophoretic location of the target polynucleotide plus an incorporated 3′-AZ-dNMP at the 3′-end; lane 3 shows the electrophoretic location of the target polynucleotide plus an incorporated 3′-AZ-dNMP coupled with, or without, a fluorescent quencher at the 3′-end; and lane 4 shows the electrophoretic location of the target polynucleotide plus an incorporated 3′-AZ-dNMP coupled with a fluorescent quencher at the 3′-end after the enrichment step.

FIG. 6 shows the electrophoretic result of an example of labeling the 3′-end of a polynucleotide having a partial double-stranded region formed by a 60-mer strand and a 20-mer strand. In FIG. 6, lane S shows the electrophoretic location of the partial double-stranded polynucleotide before a labeling reaction; lane 1 shows the result of formation of a 61-mer forward strand carrying an azide group at the 3′-end; lane 2 shows the result after the labeling reaction; and lane 3 shows the result after a clean-up reaction.

FIG. 7 shows the electrophoretic result of labeling a Cy5-fluorescent dye/fluorophore to the 3′-end of a polynucleotide through the enzymatic synthesis of different 3′-O-azidomethyl deoxynucleotides. Lane S1: the electrophoretic location of the polynucleotide before incorporating Cy5-labeled 3′-AZ-dATP; lane 1: the electrophoretic location of the Cy5-labeled 3′-AZ-dATP-incorporated polynucleotide after a labeling reaction; lane 1-1: the electrophoretic location of the Cy5-labeled polynucleotide after a clean-up reaction: lane S2: the electrophoretic location of the polynucleotide before incorporating Cy5-labeled 3′-AZ-dGTP; lane 2: the electrophoretic location of the Cy5-labeled 3′-AZ-dGTP-incorporated polynucleotide after a labeling reaction; lane 2-1: the electrophoretic location of the Cy5-labeled polynucleotide after a clean-up reaction; lane S3: the electrophoretic location of the polynucleotide before incorporating IF700-labeled 3′-AZ-dCTP; lane 3: the electrophoretic location of the IF700-labeled 3′-AZ-dCTP-incorporated polynucleotide after a labeling reaction; and lane 3-1: the electrophoretic location of the IF700-labeled polynucleotide after a clean-up reaction.

### DETAILED DESCRIPTION

The following examples are used for illustrating the present disclosure. A person skilled in the art can easily conceive the other effects of the present disclosure, based on the disclosure of the specification. It will be apparent that one or more embodiments may be practiced without specific details. The present disclosure can also be implemented or applied as described in different examples. It is possible to modify or alter the following examples for carrying out this disclosure without contravening its scope for different applications.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification.

The practice of the present disclosure employs, unless otherwise indicated, conventional techniques of molecular biology, microbiology, cell biology, biochemistry, and immunology, which are well within the purview of a skilled artisan in the art. Such techniques are explained fully in the literature, such as “Molecular Cloning: A Laboratory Manual,” second edition (Sambrook, et al., 1989), Cold Spring Harbor Press; “Oligonucleotide Synthesis” (M. J. Gait, 1984); “Methods in Molecular Biology,” Humana Press; “Cell Biology: A Laboratory Notebook” (J. E. Cellis, ed., 1998) Academic Press; “Animal Cell Culture” (R. I. Freshney, ed., 1987); “Handbook of Experimental Immunology” (Weir, 1996); “Introduction to Cell and Tissue Culture” (J. P. Mather and P. E. Roberts, 1998); “Cell and Tissue Culture: Laboratory Procedures” (A. Doyle, J. B. Griffiths, and D. G. Newell, eds., 1993-8); “Methods in Enzymology” (Academic Press, Inc.); “Handbook of Experimental Immunology” (D. M. Weir and C. C. Blackwell, eds.); “Gene Transfer Vectors for Mammalian Cells” (J. M. Miller and M. P. Calos, eds., 1987); “Current Protocols in Molecular Biology” (F. M. Ausubel, et al., eds., 1987); “PCR: The Polymerase Chain Reaction (Mullis, et al., eds., 1994); and “Current Protocols in Immunology” (J. E. Coligan et al., eds., 1991); “Short Protocols in Molecular Biology” (Wiley and Sons, 1999); “Immunobiology” (C. A. Janeway and P. Travers, 1997); “Antibodies” (P. Finch, 1997): “Antibodies: a practical approach” (D. Catty., ed., IRL Press, 1988-1989); “Monoclonal antibodies: a practical approach” (P. Shepherd and C. Dean, eds., Oxford University Press, 2000); “Using antibodies: a laboratory manual” (E. Harlow and D. Lane (Cold Spring Harbor Laboratory Press, 1999); The Antibodies (M. Zanetti and J. D. Capra, eds., Harwood Academic Publishers, 1995). Particularly useful techniques for particular embodiments will be discussed in the sections that follow. Without further elaboration, it is believed that one skilled in the art can, based on the above descriptions, utilize the present disclosure to its fullest extent. The following embodiments are, therefore, to be construed as merely illustrative, and not limitative of the remainder of the disclosure in any way whatsoever. All publications cited herein are incorporated by reference for the purposes or subject matter referenced herein.

As used herein, the singular forms “a,” “an,” and “the” are intended to include the plural forms, unless the context clearly indicates otherwise. The terms “includes,” “including,” “comprises,” and “comprising” are used in either the detailed descriptions and/or the claims, and such terms are intended to be inclusive in a manner of not excluding others, such as other components, materials, steps, etc. The terms “sec,” “min,” and “hr” as used herein are abbreviations of “second,” “minute,” and “hour.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of this disclosure, unless the context clearly dictates otherwise.

As used herein, the terms “about,” “approximately,” and “around” generally mean within 10%, 5%, 1%, or 0.5% of a given value or range. Alternatively, the term “about,” “approximately,” and “around” mean within an acceptable standard error of the mean when considered by one of ordinary skill in the art. Unless otherwise expressly specified, all of the numerical ranges, amounts, values, and percentages such as those for quantities of materials, durations of time periods, temperatures, operating conditions, ratios of amounts, and the likes disclosed herein should be understood as modified in all instances by the terms “about,” “approximately,” or “around.”

As used herein, the term “derived,” when referring to a biological sample, indicates the sample being obtained from the stated source at some point in time. For example, a biological sample derived from an organism can represent a primary biological sample obtained directly from the organism (i.e., unmodified), or can be modified, e.g., by introduction of a recombinant vector, by culturing under particular conditions, or immortalization.

The terms “nucleic acid,” “nucleic acid sequence,” and “nucleic acid fragment” as used herein refer to a nucleotide sequence in a single-stranded or double-stranded form, of which the sources are not limited herein, and generally, include naturally occurring nucleotides or artificial chemical mimics. The term “nucleotide” as used herein refers to the monomeric unit of nucleic acids or polynucleotides as described hereafter, having a glycoside with or without a nucleobase, and one or more internucleotide linkages, e.g., phosphodiester linkage. In some embodiments, the nucleobase includes naturally occurring bases such as adenine (A), thymine (T), cytosine (C), guanine (G), and uracil (U), non-naturally occurring bases such as xanthine, hypoxanthine, isoguanine, and isocytosine, as well as any analogs or derivatives thereof. In some embodiments, a nucleotide with an abasic site (loss of nucleobase) is also included within the scope of the present disclosure. In some embodiments, the sugars in the glycoside include naturally occurring sugars such as pentose sugars (e.g., deoxyribose and ribose), non-naturally occurring sugars, and the analogs thereof. In some embodiments, nucleotides are linked via internucleotide linkages such as, but not limited to, phosphate, boranephosphate, phosphorothioate, phosphodiester, phosphotriester, H-phosphonate, aminophosphonate, methylphosphonate, phosphonoacetate, sulfur phosphonoacetate, or other variants of the phosphate backbone of natural nucleic acids. The term “nucleotide” as used herein also encompasses structural analogs in place of natural or nonnatural nucleotides, such as modified nucleotides. For example, the term “xeno-nucleotide” refers to the nucleotide being modified to have a different sugar moiety than those contained in a natural DNA or RNA. The exemplary nucleic acids having the xeno-nucleotide, i.e., xeno-nucleic acids (XNA), include but are not limited to peptide nucleic acid (PNA), locked nucleic acid (LNA), 1,5-anhydrohexitol nucleic acid (HNA), threose nucleic acid (TNA), glycol nucleic acid (GNA), cyclohexene nucleic acid (CeNA), and FANA (fluoro arabino nucleic acid).

As used herein, the term “polynucleotide” refers to a polymer of nucleotides and is generic to any type of nucleic acids such as natural or non-natural DNAs or RNAs and modified nucleic acids such as xeno-nucleic acids (XNA) as described herein. A polynucleotide can also include any combinations of glycosides with or without a nucleobase and internucleotide linkages. Unless otherwise specified, the polynucleotide described herein has an intrinsic directionality in terms of the 5′-end of one nucleotide to the 3′-end of its neighboring nucleotide, where the template-independent synthesis of a polynucleotide provided herein proceeds in a 5′ to 3′ direction.

The term “polynucleotide” used herein is not intended to be distinct in length of nucleotide unit, where the term refers only to the polymeric molecule structure. That is to say, a polynucleotide used herein is interchangeable with the term “oligonucleotide” and can range in size from a few monomeric nucleotide units to several thousands of monomeric nucleotide units, such as 2 to 5 nucleotides, 5 to 20 nucleotides, 20 to 100 nucleotides, 100 to 1,000 nucleotides, or longer. A polynucleotide can be composed entirely of natural or non-natural occurring, modified or non-modified deoxyribonucleotides, entirely of natural or non-natural occurring, modified or nonmodified ribonucleotides, or chimeric mixtures thereof. Nucleobases (also known as nitrogenous bases) contained in a polynucleotide may be, for example, adenine, thymine, cytosine, guanine, uracil, xanthine, hypoxanthine, isocytosine, or isoguanine. In addition, a polynucleotide may contain one or more abasic sites (an apurinic/apyrimidinic site), also known as AP sites.

The term “initiator” as used herein refers to a nucleoside monomer, a nucleotide monomer, an oligonucleotide, a polynucleotide, or modified analogues thereof, from which a nucleic acid is to be synthesized by a nucleic acid polymerase de novo. The term “initiator” may also refer to an XNA having a 3′-hydroxyl group, such as a 3′-hydroxyl-PNA.

According to the present disclosure, the initiator may also be linked or immobilized to a solid support, and a linking nucleotide is coupled to a 3′-end nucleotide of the initiator and a 5′-end nucleotide of the synthesized nucleic acid. The initiator may be directly attached to the solid support, attached to the support via a linker, or immobilized via physical interactions such as adsorption, electrostatic interaction, and hydrogen bonds. Examples of the solid support include, but are not limited to, microarrays, beads (coated or non-coated), columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, magnetic particles, plastics (such as polyethylene, polypropylene, and polystyrene), gel forming materials (such as proteins (e.g., gelatins), lipopolysaccharides, silicates, agarose, polyacrylamides, or methyl methacrylate polymers), sol-gels, porous polymers, hydrogels, nanostructured surface nanotubes (such as carbon nanotubes), and nanoparticles (such as gold nanoparticles or quantum dots).

The term “polymerase” as used herein refers to an enzyme/protein capable of synthesizing nucleic acids, which is generically a DNA polymerase, an RNA polymerase, or a functionally equivalent enzyme, including naturally-occurring enzymes, modified enzymes, enzyme subunits and the derivatives thereof. For example, an amino acid sequence modification (e.g., mutation and functional group substitution) can be applied to these enzymes for desired purposes, such as removing the 5′ to 3′ exonuclease activity and enhancing the polymerase activity, to obtain altered enzymes with improved properties, such as thermostability/thermotolerance and catalytic efficiency.

According to the present disclosure, the term “polymerase” may be a template dependent polymerase or a template-independent polymerase. The polymerase may include a family-A DNA polymerase (e.g., T7 DNA polymerase, Pol I, Pol γ, θ, and ν), a family-B DNA polymerase (e.g., Pol II, Pol B, Pol ζ, Pol α, δ and ε), a family-C DNA polymerase (e.g., Pol III), a family-D DNA polymerase (e.g., PoID), a family-X DNA polymerase (e.g., Pol β, Pol σ, Pol λ, Pol μ and terminal deoxynucleotidyl transferase (TdT)), a family-Y DNA polymerase (e.g., Pol τ, Pol κ, Pol η, DinB, Pol IV and Pol V), a reverse transcriptase (e.g., telomerase and hepatitis B virus reverse transcriptase), and enzymatically active fragments thereof.

Non-limiting examples of widely employed template-dependent polymerases include T7 DNA polymerase of T7 bacteriophage and T3 DNA polymerase of T3 bacteriophage, which are DNA-dependent DNA polymerases; T7 RNA polymerase of T7 bacteriophage and T3 RNA polymerase of T3 bacteriophage, which are DNA-dependent RNA polymerases; DNA polymerase I or its fragment known as the Klenow fragment of Escherichia col, which is a DNA-dependent DNA polymerase; Thermophilus aquaticus DNA polymerase, Tth DNA polymerase and Vent DNA polymerase, which are thermostable DNA-dependent DNA polymerases; eukaryotic DNA polymerase β, which is a DNA-dependent DNA polymerase; telomerase, which is an RNA-dependent DNA polymerase; and non-protein catalytic molecules, such as modified RNA (ribozymes: Unrau & Bartel, 1998) and DNA with template-dependent polymerase activity.

Non-limiting examples of the template-independent polymerases include reverse transcriptase, poly A polymerase, DNA polymerase theta (θ), terminal deoxynucleotidyl transferase (TdT), and DNA polymerase mu (μ). Since polymerases suitable for performing nucleic acid synthesis, nucleotide addition/incorporation, and process of nucleic acid synthesis are within the expertise and routine skills of those skilled in the art, further details thereof are omitted herein for the sake of brevity. Furthermore, the B-family DNA polymerases provided previously by inventors are also suitable for being used under template-independent conditions, and the U.S. Ser. No. 11/591,629B2 is hereby incorporated entirely by reference.

The term “modification” as used herein refers to the alteration(s) of the chemical structure of a reactant molecule. When a nucleic acid is used as a reactant molecule, the means of modifications include, but are not limited to, the introduction of an additional chemical group/moiety to the nucleic acid, removal or substitution of an original chemical group/moiety from the nucleic acid, or the combination thereof, regardless of the source of the nucleic acid. Alternatively, the modification(s) may be introduced to a specific sequence of nucleic acid during the de novo nucleic acid synthesis resulting in a direct modification, or modifications, on the nucleic acid. For example, a fluorophore-labeled nucleotide analogue can be incorporated into a nucleic acid alongside with natural counterparts to become a “fluorescent labeled” nucleic acid. Likewise, a site-specific modification, or modifications, can also be inserted enzymatically into a nucleic acid by incorporating nucleotide(s) carrying desired modification(s). For example, a nucleoside triphosphate having a 3′-O-azidomethyl group can be enzymatically introduced to the 3′-end of a nucleic acid, thereby directly adding an azidomethyl modification to the 3′-end of a nucleic acid. Such modifications result in the addition of a nucleotide together with a site-specific chemical group to a target nucleic acid.

As used herein, the term “detecting” or “detection” refers to both quantitative and qualitative determinations and as such, the term “detecting” or “detection” is used interchangeably herein with “assaying,” “measuring,” and the like. Where a quantitative determination is intended, the phrase “determining an amount” and the like is used. Where either a qualitative or quantitative determination is intended, the phrase “determining a level” or “detecting a level” may be used.

Although the present disclosure is illustrated by specific embodiments and optional features, it is understood that modifications and variations of the concepts herein disclosed may be resorted to by those skilled in the art, and that such modifications and variations are considered to be within the scope of the present disclosure.

### Example

Exemplary embodiments according to the present disclosure are further described in the following examples, which should not be construed to limit the scope of the present disclosure.

Provided herein are examples of modifying nucleic acids using the method of the present disclosure, and several exemplary modifications of polynucleotides are demonstrated to produce a labeled nucleic acid with a fluorophore or quencher at its 3′-end.

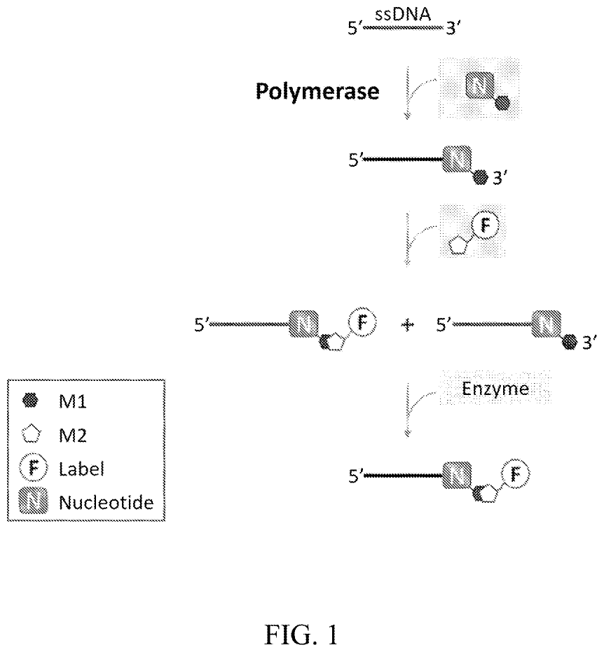

As illustrated in FIG. 1, the methods used in these modifications can be practically divided into three sequential steps: 1) using a polymerase to incorporate a modified nucleotide carrying a reactive moiety to the 3′-end of a target polynucleotide; 2) conjugating a desired molecule having a label moiety such as a fluorescent dye or a quencher and a corresponding functional moiety to the 3′-end of the target polynucleotide; 3) degrading unlabeled or incompletely labeled polynucleotides by a 3′ to 5′ exonuclease to enrich the desired, labeled polynucleotides.

In some embodiments, an enzymatic synthesis approach is used to introduce the reactive moiety to the target polynucleotide. For example, a nucleotide analogue, or analogues, is enzymatically added to the 3′-hydroxyl (3′-OH) end of a single-stranded nucleic acid initiator (the target polynucleotide) in a template-independent synthesis manner to produce a polynucleotide with a desired reactive moiety. In at least one embodiment, the desired reactive moiety is an azide (N₃) or azido group, and a suitable reagent/compound, such as a nucleotide analogue, containing an azido moiety, such as a 3′-O-azidomethyl group, may be used to introduce such a modification to the 3′-end of polynucleotides. The following examples are further provided based on this scenario.

### Example 1: Preparation of a Polynucleotide Containing a 3′-O-Azidomethyl Group

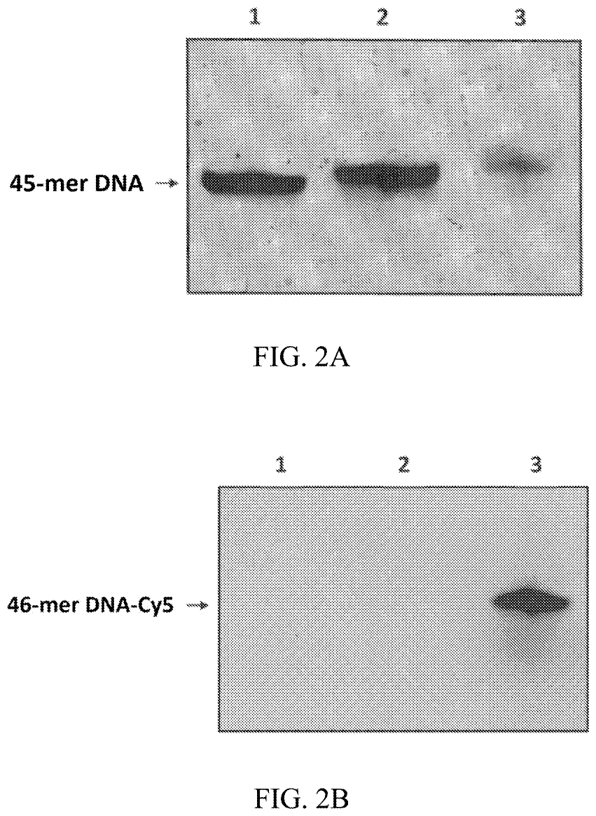

A 45-mer DNA polynucleotide containing a fluorescein label at the 5′-end (5′-FAM-45-mer DNA) was used as the target polynucleotide for 3′-modification or labeling. To introduce an azidomethyl group to the 3′-end of the 5′-FAM-45-mer DNA, the 3′-O-azidomethyl-deoxynucleoside triphosphate (3′-AZ-dNTP) was used as a template-independent DNA synthesis substrate for the selective B-family DNA polymerase or its variant. The exemplary polymerase used herein may be an in-house polymerase variant derived from Vent DNA polymerase, which can efficiently incorporate the 3′-AZ-dNTP to the 3′-end of the target polynucleotide. The nucleotide incorporation reaction was performed in the reaction mixture (10 μL) containing 100 nM of 5′-FAM-45-mer DNA target polynucleotide, 0.25 mM manganese chloride (MnCl₂), and 200 nM of polymerase. The reaction was initiated by the addition of 25 μM of 3′-O-azidomethyl-dTTP (3′-AZ-dTTP) and then incubated at 60° C. for a designated period of time. Time periods from 2 minutes to 30 minutes have been used. The reaction was then terminated by adding a 10 μL of 2×quench solution (95% de-ionized formamide and 25 mM EDTA). The reaction mixture was further denatured at 95° C. for 10 min, and the reaction products were analyzed by 20% polyacrylamide gel electrophoresis containing 8 M urea (Urea-PAGE). The reaction products were then visualized by imaging the gel on the Amersham Typhoon Laser Scanner (Cytiva Life Sciences, Marlborough, MA, USA). At the end of the polymerase-dependent 3′-AZ-dTMP incorporation reaction to the 5′-FAM-45-mer DNA, it resulted in the formation of a 5′-FAM-46-mer DNA carrying an azide group at the 3′-end (shown as lane 2 of FIGS. 2A, 3, and 5, respectively), which can be used for a subsequent labeling reaction. Alternatively, the 5′-FAM-46-mer DNA was further purified using the DNA QIAquick Nucleotide Removal Kit (Qiagen, Germantown, MD) before the subsequent labeling reaction. The preparation of a 3′-azide-labeled target polynucleotide containing a 3′-O-azidomethyl group is shown in Scheme 1 below.

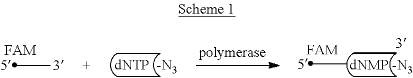

### Example 2: 3′—Labeling of a Polynucleotide Carrying an Azidomethyl Group at the 3′-End Via an Azide-Alkyne Coupling Reaction

As previously described in Example 1, once the polynucleotide containing an azidomethyl group at the 3′-end is obtained, the 3′-end azide (N₃) group can be directly used for labeling with any desired tag or functional molecule, such as a fluorescent dye or quencher, via an azide-alkyne coupling reaction.

For example, when a Cyanine 5 (Cy5) fluorophore-label at the 3′-end of the target polynucleotide is desired, the Cy5-alkyne molecule can be chosen for the azide-alkyne coupling reaction with the polynucleotide carrying an azide (N₃) group at the 3′-end. In at least one embodiment, the polynucleotide with a 3′-azide group can directly react with the Cy5-alkyne in the presence of a tris(3-hydroxypropyltriazolylmethyl)amine (THPTA) ligand, copper ions, and sodium ascorbate to trigger the Cu-catalyzed cycloaddition reaction between the 3′-azide group of the polynucleotide and the alkyne group of the Cy5-alkyne molecule. The azide-alkyne coupling reaction was normally performed at 37° C. for 1 hour. The unreactive Cy5-alkyne molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′ exonuclease) treatment at 37° C. for 1 hour. The final reaction products were analyzed with 20% Urea-PAGE and visualized by imaging the gel on the Amersham Typhoon Laser Scanner (Cytiva Life Sciences, Marlborough, MA, USA). The results of azide-alkyne cycloaddition and enzyme-digestion reactions generate a homogeneous target polynucleotide containing a Cy5 fluorophore at the 3′-end (referring to lane 3 of FIGS. 2A and 2B). The labeling of a Cy5 fluorophore to the 3′-end of a target polynucleotide is shown in Scheme 2 below.

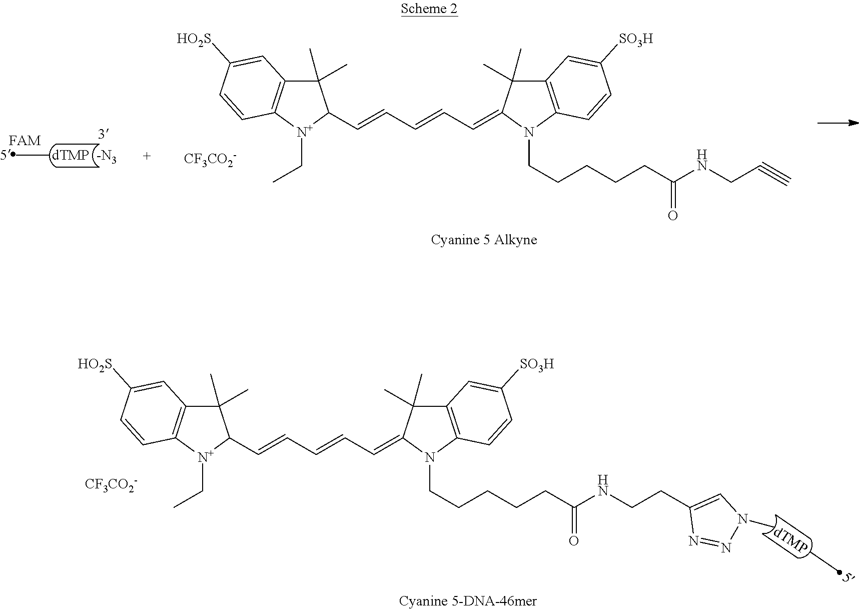

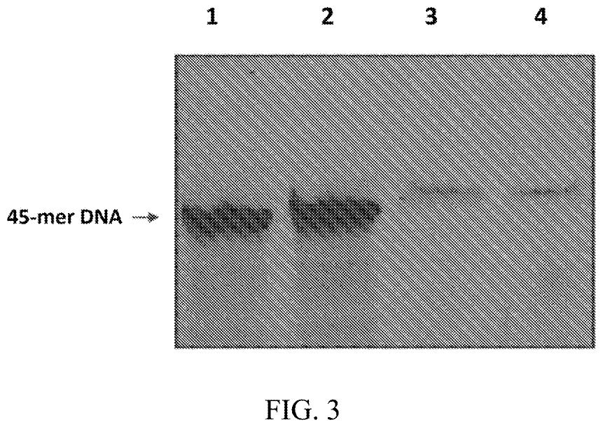

Similarly, when a fluorescent quencher label, such as a Black Hole Quencher 1 (BHQ1), at the 3′-end of a target polynucleotide is desired, the BHQ1-alkyne molecule can be chosen for the azide-alkyne coupling reaction with the target polynucleotide carrying an azide (N₃) group at the 3′-end. For example, the polynucleotide with a 3′-azide group was reacted with the BHQ1-alkyne in the presence of a tris(3-hydroxypropyltriazolylnethyl)amine (THPTA) ligand, copper ions, and sodium ascorbate to trigger the Cu-catalyzed cycloaddition reaction between the 3′-azide group and the alkyne group of the BHQ1-alkyne molecule. The azide-alkyne coupling reaction was normally performed at 37° C. for 1 hour. The unreactive BHQ1-alkyne molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′exonuclease) treatment at 37° C. for 1 hour. The final reaction products were analyzed with 20% Urea-PAGE and visualized by imaging the gel on the Amersham Typhoon Laser Scanner (Cytiva Life Sciences, Marlborough, MA, USA). The sequential azide-alkyne cycloaddition and enzyme-digestion reactions generate a homogeneous target polynucleotide containing a BHQ1 label at the 3′-end (referring to lane 4 of FIG. 3). The labeling of a fluorescent quencher to the 3′-end of a target polynucleotide is shown in Scheme 3 below.

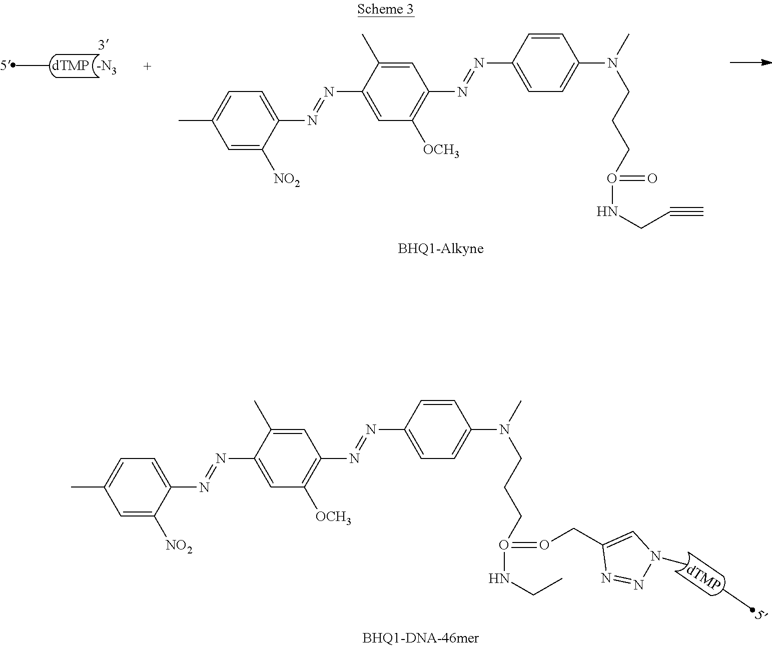

The components and individual experimental groups of the BHQ1-labeling reaction are summarized in Table 1 below. The results of the labeling reaction corresponding to each experimental group are shown in lanes 1 to 4 in FIG. 3, respectively. In Table 1, four experimental groups were designed, and the reaction components are shown, in which the symbol “+” denotes the addition of a designated reagent to the reaction in each experimental group, and the symbol “−” denotes that the designated reagent is not added to the reaction in the experimental group.

TABLE 1

Experimental
groups

| Components | (1) | (2) | (3) | (4) |
|---|---|---|---|---|
| 5′-FAM-45-mer DNA polynucleotide | + | + | + | + |
| DNA polymerase | − | + | + | + |
| 3′-AZ-dTTP | − | + | + | + |
| BHQ1-alkyne | − | − | + | + |
| 3′ to 5′ exonuclease | − | − | − | + |

### Example 3: 3′—Labeling of a Polynucleotide Carrying an Azide Group at the 3′-End Via an Azide-DBCO Ligation Reaction

Alternatively, once the polynucleotide having an azidomethyl group at the 3′-end is obtained, the terminal azide group can also be used for labeling with any desired tag or functional molecule, such as a fluorescent dye or quencher, via the azide-dibenzoazacyclooctyne (DBCO) ligation reaction.

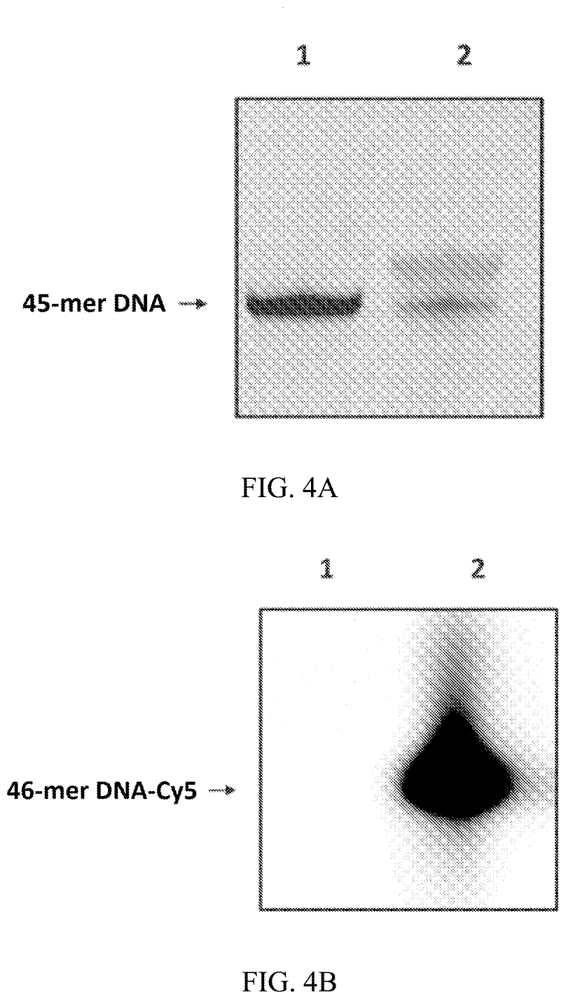

For example, when a Cyanine 5 (Cy5) fluorophore-label at the 3′-end of a target polynucleotide is desired, the Cy5-DBCO molecule can be chosen for the azide-DBCO ligation reaction with the polynucleotide carrying an azide (N₃) group at the 3′-end. In at least one embodiment, the polynucleotide with a 3′-azide group can directly react with the Cy5-DBCO in the 1×TE buffer. The azide-DBCO ligation reaction was normally performed at 37° C. for 1 hour. The unreactive Cy5-DBCO molecule was then removed by using the DNA QIAquick Nucleotide Removal Kit (Qiagen, Germantown, MD, USA). The reaction products were analyzed with 20% Urea-PAGE and visualized by imaging the gel on the Amersham Typhoon Laser Scanner (Cytiva Life Sciences, Marlborough, MA, USA). The azide-DBCO ligation reaction generates a target polynucleotide having a Cy5 fluorophore at the 3′-end (referring to lane 2 of FIGS. 4A and 4B). The labeling of a Cy5 fluorophore to the 3′-end of a target polynucleotide is shown in Scheme 4 below.

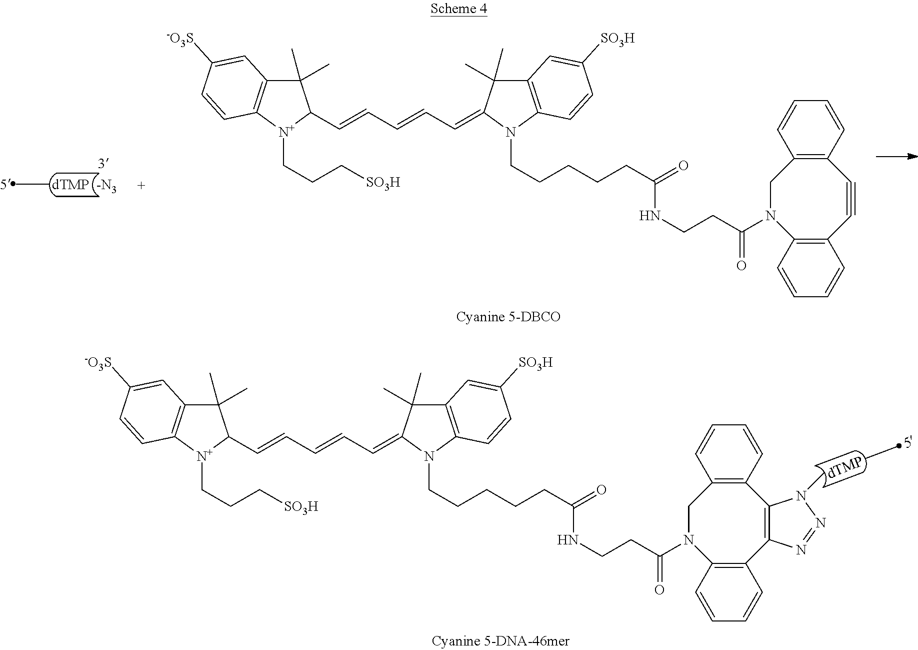

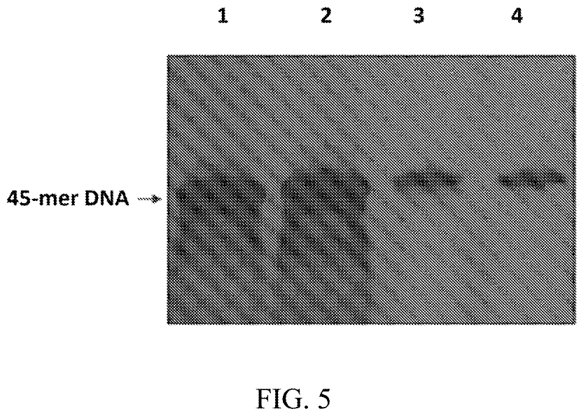

Similarly, when a fluorescent quencher label, such as a Black Hole Quencher 1 (BHQ1) at the 3′-end of a polynucleotide is desired, the BHQ1-DBCO molecule can be used for the azide-DBCO ligation reaction with the target polynucleotide carrying an azide (N₃) group at the 3′-end. For example, the target polynucleotide with a 3′-azide group can directly react with the BHQ1-DBCO in the 1×TE buffer. The azide-DBCO ligation reaction was normally performed at 37° C. for 1 hour. The unreactive BHQ1-DBCO molecule was then removed by using the DNA QIAquick Nucleotide Removal Kit (Qiagen, Germantown, MD, USA). The clean-up reaction products were further subjected to the 3′ to 5′ exonuclease (e.g., 200 nM of 3′ to 5′ exonuclease) treatment at 37° C. for 1 hour. The final reaction products were analyzed with 20% Urea-PAGE and visualized by imaging the gel on the Amersham Typhoon Laser Marlborough, MA, USA). The sequential azide-DBCO ligation and enzyme-digestion reactions generate a homogeneous target polynucleotide having a BHQ1 label at the 3′-end (referring to lane 4 of FIG. 5). The labeling of a fluorescent quencher to the 3′-end of a target polynucleotide is shown in Scheme 5 below.

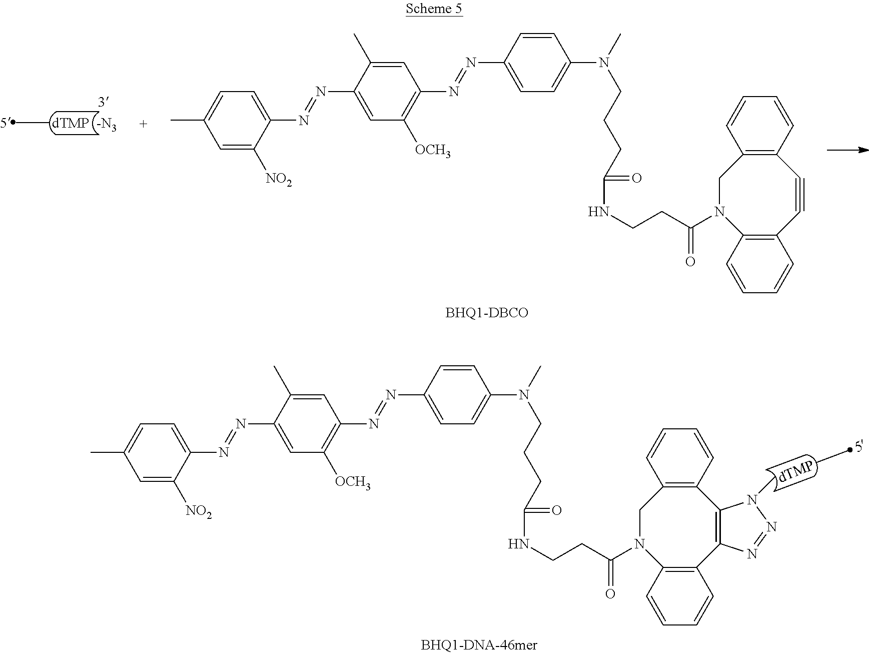

The components and individual experimental groups of BHQ1-labeling reaction are summarized in Table 2. The results of the labeling reaction corresponding to each experimental group are shown in lanes 1 to 4 in FIG. 5, respectively. In Table 2, four experimental groups were designed, and the reaction components are shown, in which the symbol “+” denotes the addition of a designated reagent to the reaction in each experimental group, and the symbol “−” denotes that the designated reagent is not added to the reaction in the experimental group.

TABLE 2

Experimental
groups

| Components | (1) | (2) | (3) | (4) |
|---|---|---|---|---|
| 5′-FAM-45-mer DNA polynucleotide | + | + | + | + |
| (5′-FAM-labeled 45-mer DNA) | | | | |
| DNA polymerase | − | + | + | + |
| 3′-AZ-dTTP | − | + | + | + |
| BHQ1-DBCO | − | − | + | + |
| 3′ to 5′ exonuclease | − | − | − | + |

### Example 4: 3′—Labeling of a Polynucleotide Having a Partial Double-Stranded Region

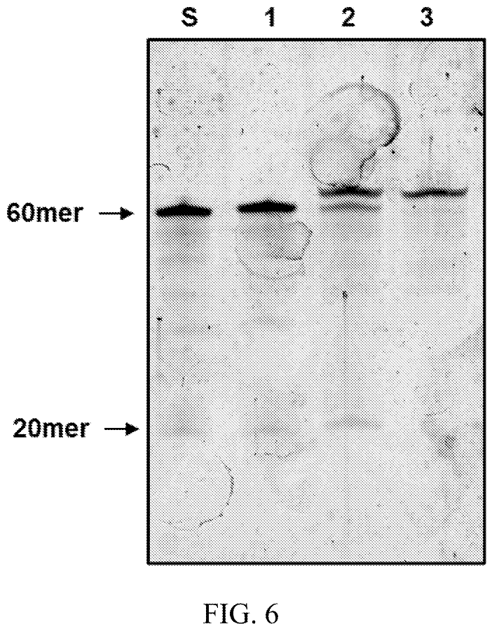

A polynucleotide having a partial double-stranded region can also be used as the target polynucleotide for 3′-modification or labeling by adding a 3′-AZ-dNTP to the 3′-end of the target polynucleotide. For example, a partial double-stranded polynucleotide consisting of a 60-mer forward strand and a 20-mer reverse strand was used as the target polynucleotide. To introduce an azidomethyl group to the 3′-end of the partial double-stranded DNA, the 3′-O-azidomethyl-deoxynucleoside triphosphate (3′-AZ-dNTP) was used as a template-independent DNA synthesis substrate for the selective B-family DNA polymerase or its variant. The nucleotide incorporation reaction was performed in the reaction mixture (10 μL) containing 100 nM of the partial double-stranded target polynucleotide, 0.25 mM of manganese chloride (MnCl₂), and 200 nM of the polymerase. The reaction was initiated by the addition of 25 μM of 3′-O-azidomethyl-dTTP (3′-AZ-dTTP) and then incubated at 60° C. for 10 minutes. The reaction was then terminated by adding a 10 μL of 2×quench solution (95% de-ionized formamide and 25 mM EDTA). The reaction mixture was further denatured at 95° C. for 10 min, which was then subjected to 20% polyacrylamide gel electrophoresis containing 8 M urea (Urea-PAGE). The reaction products were visualized by imaging the gel on the Amersham Typhoon Laser Scanner (Cytiva Life Sciences, Marlborough, MA, USA). After the polymerase-dependent 3′-AZ-dTMP incorporation reaction to the partial double-stranded polynucleotide, formation of a 61-mer forward strand carrying an azide group at the 3′-end was obtained (shown as lane 1 of FIG. 6, wherein lane S shows the electrophoretic location of the partial double-stranded polynucleotide before the 3′-end nucleotide incorporation reaction), which can be used for a subsequent labeling reaction.

In this example, the partial double-stranded polynucleotide containing an azidomethyl group at the 3′-end as obtained above was subjected to a subsequent labeling reaction via an azide-dibenzoazacyclooctyne (DBCO) ligation, as mentioned above.

The result of the labeling reaction of the partial double-stranded polynucleotide is shown in FIG. 6, wherein lane S shows the electrophoretic location of the partial double-stranded polynucleotide before a labeling reaction; lane 1 shows the result of formation of a 61-mer forward strand carrying an azide group at the 3′-end; lane 2 shows the result after the labeling reaction, and lane 3 shows the result after a clean-up reaction, in which the labeled products were further subjected to the 3′ to 5′ exonuclease (e.g., 200 nM of 3′ to 5′exonuclease) treatment at 37° C. for 1 hour.

This example demonstrates the applicability of the 3′-labeling method of the present disclosure to a polynucleotide having a partial double-stranded region, or a DNA consisting of strands of different lengths.

### Example 5: 3′—Labeling of a Polynucleotide with Different 3′-O-Azidomethyl Deoxynucleotides

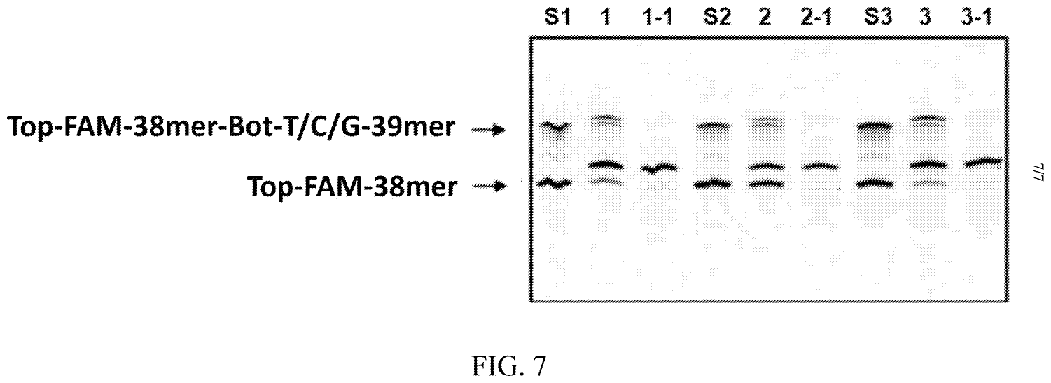

A 38-mer DNA polynucleotide containing a fluorescein label at the 5′-end (5′-FAM-38-mer DNA) was used as the target polynucleotide for 3′-modification or labeling. To introduce an azidomethyl group to the 3′-end of the 5′-FAM-38-mer DNA, different fluorescent-labeled 3′-O-azidomethyl-deoxynucleoside triphosphates including Cy5-labeled 3′-AZ-dATP, Cy5-labeled 3′-AZ-dGTP and IF700-labeled 3′-AZ-dCTP were used as a template-independent DNA synthesis substrate for the selective B-family DNA polymerase used. The exemplary polymerase used herein is a Vent polymerase, which can efficiently incorporate the various 3′-AZ-dNTP to the 3′-end of the target polynucleotide. The nucleotide incorporation reactions were performed in the reaction mixtures (10 μL) containing 100 nM of 5′-FAM-38-mer DNA target polynucleotide, 0.25 mM of manganese chloride (MnCl₂), and 200 nM of polymerase. The reactions were initiated by the addition of 25 M of Cy5-labeled 3′-AZ-dATP, Cy5-labeled 3′-AZ-dGTP or IF700-labeled 3′-AZ-dCTP, respectively, and then incubated at 60° C. for 20 minutes. The reactions were then terminated by adding a 10 μL of 2×quench solution (95% de-ionized formamide and 25 mM EDTA) to each reaction mixture, which were further denatured at 95° C. for 10 min. The reaction products were then analyzed by 20% polyacrylamide gel electrophoresis containing 8 M urea (Urea-PAGE) and visualized by imaging the gel on the Amersham Typhoon Laser Scanner (Cytiva Life Sciences, Marlborough, MA, USA). After incorporating the Cy5-labeled 3′-AZ-dATP, Cy5-labeled 3′-AZ-dGTP and IF700-labeled 3′-AZ-dCTP to the 5′-FAM-38-mer polynucleotides, 5′-FAM-39-mer polynucleotides were formed, carrying an azide group at the 3′-end (shown as lanes 1, 2 and 3 of FIG. 7, respectively).

In this example, the 5′-FAM-39-mer polynucleotides containing different azidomethyl groups at the 3′-end as obtained above were subjected to a subsequent labeling reaction via a direct incorporation of fluorescent-labeled 3′-O-azidomethyl-deoxynucleoside triphosphates.

The result of the labeling reaction of the partial double-stranded polynucleotide is shown in FIG. 7, wherein lanes S1, S2 and S3 show the electrophoretic locations of the 5′-FAM-38-mer and 5′-FAM-39-mer polynucleotides before a labeling reaction, respectively; and lanes 1, 2 and 3 show the electrophoretic locations of the fluorescent-labeled 5′-FAM-38-mer and 5′-FAM-39-mer polynucleotides after the labeling reaction, respectively; and lanes 1-1, 2-1 and 3-1 show the results after a clean-up reaction, in which the labeled products were further subjected to the 3′ to 5′ exonuclease (e.g., 200 nM of 3′ to 5′exonuclease) treatment at 37° C. for 1 hour.

This example demonstrates the applicable uses of different 3′-O-azidomethyl-deoxynucleoside triphosphates to introduce an azidomethyl group to the 3′-end of a target polynucleotide using the 3′-labeling method of the present disclosure.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

1: A kit for modifying a 3′-end of a polynucleotide, comprising:
a nucleotide having a reactive moiety;
a polymerase to incorporate the nucleotide having the reactive moiety to the 3′-end of the polynucleotide; and
a desired molecule having a corresponding functional moiety capable of reacting with the reactive moiety.

2: The kit of claim 1, wherein the desired molecule further comprises a label moiety to form a labeled polynucleotide when the desired molecule is coupled to the 3′-end of the polynucleotide.

3: The kit of claim 1, wherein the polymerase is a template-independent polymerase.

4: The kit of claim 1, wherein the polymerase is a DNA polymerase, an RNA polymerase, or a functionally equivalent enzyme thereof.

5: The kit of claim 4, wherein the DNA polymerase is an A family DNA polymerase, a B family DNA polymerase, or an X family DNA polymerase.

6: The kit of claim 5, wherein the B family DNA polymerase is a Thermococcaceae DNA polymerase.

7: The kit of claim 6, wherein the B family DNA polymerase is a Thermococcus DNA polymerase or a Pyrococcus DNA polymerase.

8: The kit of claim 7, wherein the B family DNA polymerase is selected from the group consisting of a B family DNA polymerase of Thermococcus kodakarensis, a B family DNA polymerase of Pyrococcus furiosus, a B family DNA polymerase of Thermococcus litoralis, a B family DNA polymerase of Thermococcus sp. 9° N, and a B family DNA polymerase of Thermococcus gorgonarius.

9: The method kit of claim 4, wherein the DNA polymerase is a modified DNA polymerase.

10. (canceled)

11: The kit of claim 1, wherein the polynucleotide is linked to an initiator attached to a solid support, and the kit further comprises an endonuclease to enzymatically release the polynucleotide from the initiator.

12. (canceled)

13: The kit of claim 1, wherein the nucleotide incorporated to the 3′-end of the polynucleotide is a natural nucleotide, a nucleotide analogue, or an abasic nucleotide.

14: The method kit of claim 1, wherein the corresponding functional moiety is capable of reacting with the reactive moiety via a bioorthogonal reaction.

15: The kit of claim 14, wherein the bioorthogonal reaction is click conjugation, oxime/hydrazone formation, Staudinger ligation, tetrazine ligation, or quadricyclane ligation.

16: The kit of claim 15, wherein the click conjugation is selected from the group consisting of copper-catalyzed azide-alkyne cycloaddition, strain-promoted azide-alkyne cycloaddition, isocyanide-based click reaction, and inverse electron demand Diels-Alder reaction.

17: The kit of claim 1, wherein the desired molecule is molecularly recognizable through detection of visible light, fluorescence, photoluminescence, electrochemiluminescence, laser, irradiation, fluorescence resonance energy transfer, fluorogenic conformational change, or fluorescence quenching.

18: The kit of claim 17, wherein the desired molecule is a chemical compound, a fluorescent tag, a dye, a marker, a reporter, a quencher, an amine, an antigen, a ligand, a protein, an antibody, an antibody fragment, a peptide, a peptide analog, or a quantum dot.

19. (canceled)

20: A method for introducing a modification to a 3′-end of a polynucleotide, comprising:
providing a nucleotide having a reactive moiety;
incorporating the nucleotide having the reactive moiety to the 3′-end of the polynucleotide;
providing a desired molecule having a corresponding functional moiety capable of reacting with the reactive moiety; and
exposing the polynucleotide to the desired molecule to form a linkage between the reactive moiety and the corresponding functional moiety, thereby coupling the desired molecule to the 3′-end of the polynucleotide.

21: The method of claim 20, wherein the nucleotide having the reactive moiety is incorporated to the 3′-end of the polynucleotide by a B family DNA polymerase.

22: The method according to claim 21, wherein the corresponding functional moiety reacts with the reactive moiety via click conjugation.

23: The method of claim 20, further comprising:
preparing the polynucleotide in a solution phase; and
providing a 3′ to 5′ exonuclease to digest unsuccessfully coupled or unmodified polynucleotides.
